SEQUENCE LISTING (8 sequences)

SEQ ID NO: 1 - protein, 28 aa, Lyssavirus rabies
TIWMPENPRPGTPCDIFTNSRGKRASNG

SEQ ID NO: 2 - protein, 41 aa, synthetic construct
YTIWMPENPRPGTPCDIFTNSRGKRASNGGGGRRRRRRRRR

SEQ ID NO: 3 - skipped

SEQ ID NO: 4 - protein, 16 aa, synthetic construct
MTTRTYFTPYRMKVRK

SEQ ID NO: 5 - protein, 16 aa, synthetic construct
MTTRTYYTPTRMKVSK

SEQ ID NO: 6 - protein, 17 aa, synthetic construct
MTWCDYFTPSRGKVRKS

SEQ ID NO: 7 - protein, 16 aa, synthetic construct
MTTRTYFTPYRGKVRK

SEQ ID NO: 8 - protein, 9 aa, Homo sapiens
CRTIGPSVC

(19) United States
(12) Patent Application Publication
(10) Pub. No.: US 2026/0355286 A1
(43) Pub. Date: Nov. 12, 2026

(54) BLOOD-BRAIN BARRIER TRANSMISSION OF GLUCAGON-LIKE PEPTIDE-1 AND GLUCOSE-DEPENDENT INSULINOTROPIC POLYPEPTIDE RECEPTOR AGONISTS

(71) Applicant: Neuronasal, Inc., Wexford, PA (US)

(72) Inventors: Douglas A. Greene, Basking Ridge, NJ (US); Thomas I. Bradshaw, Wynnewood, PA (US)

(21) Appl. No.: 19/666,893

(22) Filed: May 4, 2026

Related U.S. Application Data
(60) Provisional application No. 63/799,845, filed on May 5, 2025.

(51) Int. Cl. A61K 38/26; A61K 45/06; A61K 47/69; C07K 14/705; C12N 7/00

(52) U.S. Cl. CPC A61K 38/26; A61K 45/06; A61K 47/6901; C07K 14/705; C12N 7/00; C12N 2760/20142

(57) ABSTRACT

Methods of treating metabolic diseases such as diabetes and obesity as well as central nervous system diseases, such as Parkinson's disease, with glucagon-like peptide-1 and glucose-dependent insulinotropic polypeptide receptor agonists modified to transit the blood brain barrier are disclosed. The receptor agonists can include a conjugated transcytotic carrier, such as a transporter peptide, to facilitate transport through the blood brain barrier. Methods of manufacturing the modified glucagon-like peptide-1 and glucose-dependent insulinotropic polypeptide receptor agonists are further disclosed.

### CROSS-REFERENCE TO RELATED APPLICATIONS

The present application claims the priority benefit of U.S. Provisional Patent App. Ser. No. 63/799,845, filed May 5, 2025, which is hereby incorporated herein by reference in its entirety.

### REFERENCE TO SEQUENCE LISTING SUBMITTED ELECTRONICALLY

The sequence listing of the present application is submitted electronically via Patent Center as an ST.26 XML formatted sequence listing with the file name “0823534_SL.xml”, with a creation date of Jun. 18, 2026, and a file size of 7,491 bytes. This sequence listing is incorporated herein by reference in its entirety.

### BACKGROUND

The blood-brain barrier (“BBB”) refers to the selective semi-permeable membrane present between the blood and the interstitiuam of the brain which includes cerebral blood vessels that can restrict molecules and ions from passing from the blood into the brain. The BBB protects the brain from potentially harmful compounds suspended in blood from disrupting the brain. However, in addition to blocking harmful compounds, the BBB can also prevent therapeutic drugs from reaching receptors in the brain.

### DETAILED DESCRIPTION

As will be described herein, modified glucagon-like-peptide-1 receptor agonists (“GLP-IrA's”) and glucagon-like-peptide-1 and glucose-dependent insulinotropic polypeptide receptor dual agonists (“GLP-1/GIPrA's”), or pharmaceutically-acceptable salts thereof, effective for treatment of metabolic diseases and central nervous system (“CNS”) diseases are disclosed. The modified GLP-1rA's and GLP-1/GIPrA's include a conjugated transcytotic carrier to enable the modified GLP-1rA's and GLP-1/GIPrA's to transit through the BBB.

As can be appreciated, glucagon-like-peptide-1 (“GLP-1”) and glucose-dependent insulinotropic polypeptide “(GIP”), also known as gastric inhibitory polypeptide, are secreted in response to meal associated stimuli, bind to their respective GLP-1 and GIP receptors, and thereby influence metabolic-related functions including gastrointestinal (“GI”) and gastro-hormonal function (including insulin secretion and insulin sensitivity) and meal related behaviors. GLP-IrA's and GLP-1/GIPrA's are approved for the treatment of obesity and type-2 diabetes mellitus (“T2DM”) by virtue of their receptor-mediated effects on GI and gastro-hormonal action and on feeding-related reward behavior. Known GLP-1rA agonists including dulaglutide, exenatide, liraglutide, albiglutide, lixisenatide, semaglutide, and tirzepatide act on GLP-1 and GIP receptors widely expressed throughout peripheral tissues.

In addition to being expressed in peripheral tissues, GLP-1 and GIP receptors are also widely expressed within the brain. GLP-1, due to its small size of 30 to 31 amino acids, can readily diffuse across the BBB by saturable receptor-mediated transcytosis. However, GLP-1 has a biological half-life of only about 1.5 to about 5 minutes due to rapid degradation by endogenous enzymes dipeptidyl-peptidase-IV (“DPP-4”) and neutral endopeptidase (“NEP”) making GLP-1, and the related GIP, unsuitable for therapeutic applications as dosing would be required multiple times per day.

For therapeutic applications, GLP-1rA's and GLP-1/GIPrA's have been developed that exhibit longer biological half-lives. For example, therapeutic GLP-1rA's have been developed that modify GLP-1 to include one or more of modification of the 8 amino acid to prevent cleavage by DPP-4; attachment of a fatty acid chain to allow attachment to serum albumin to protect from DPP-4 and reduce renal filtration; and fusion to large proteins or domains such as the Fc domain of IgG antibodies or human serum albumin to delay renal clearance and reduce proteolysis. Although such GLP-1rA's are already useful for treatment of metabolic disorders including obesity and diabetes, they are substantially or entirely blocked by the BBB, possibly reflecting stearic restrictions or size-exclusion limitations of GLP-1-receptor mediated transcytotic capacity.

Synthetic GLP-1rA's that have been structurally modified to achieve longer plasma half-life to minimize required therapeutic dosing frequency may less globally and efficiently transit the BBB, so that the central nervous system (“CNS”) component of their feeding-related behavioral effects may primarily reflect either indirect peripheral neural input to the brain e.g. via the vagus nerve, or limited regional entry via specialized circumventricular organs (CVO's) confined to the base of the brain.

The modified GLP-1rA's or GLP-1/GIPrA's, or a pharmaceutically-acceptable salt thereof, disclosed herein can transit the BBB and provide activation of the GLP and GIP receptors expressed throughout the brain improving therapeutic outcomes associated with metabolic diseases. For example, enhanced selective entry into the brain can optimize GLP-1rA's or GLP-1/GIPrA's, for weight-loss and related metabolic indications by maximizing feeding behavior-related CNS activity while minimizing dose-limiting peripheral gastrointestinal (GI) effects responsible for treatment-related nausea, vomiting, diarrhea, and constipation.

In addition to therapeutic treatment of metabolic diseases such as obesity and diabetes, epidemiologic, preclinical, and clinical evidence suggests that GLP-1rA's and GLP-1/GIPrA's can further be efficacious in the prevention and/or treatment of CNS neurodegenerative disorders such as Parkinson's disease (“PD”) and other neurodegenerative, neuropsychiatric, traumatic, and ischemic brain diseases. Treatment of such CNS neurodegenerative disorders with current GLP-IrA's and GLP-1/GIPrA's is limited as the receptors for CNS neurodegenerative disorders are largely located in the brain.

The GLP-1 axis is associated with the modulation of inflammation both for CNS disorders and metabolic disorders, such as diabetes or obesity. Multiple naturally occurring or synthetic GLP-1rA's are approved for the treatment of patients with type 2 diabetes and/or obesity. GLP-1r stimulation enhances DNA repair via apurinic/apyrimidinic endonuclease 1 (“APE1”) which is upregulated by oxidative stress generally, and brain oxidative stress injury is tightly connected to the competence of DNA repair. APE1 also has a redox effector role in promoting the DNA-binding activity of activator protein 1 (AP-1), p53, nuclear factor-B (NF-B), hypoxia-inducible factor 1 (HIF-1), and paired box 5 (PAX5). GLP-1 increases brain glucose uptake and hexokinase activity in the human brain.

CNS GLP-1r's are thought to mediate not only the CNS's regulation of appetite, but also the CNS's modulation of systemic inflammation possibly through recently appreciated localized neuronal co-expression of GLP-1r's with al-adrenergic and d-opioid receptors.

For efficient delivery to the GLP-1 and GIP receptors in the brain, passage of GLP-1rA's or GLP-1/GIPrA's through the BBB is required such as through transporter-mediated transcytosis (e.g., receptor-mediated transcytosis), cell-mediated transcytosis, lipophilic pathways, efflux pumps, adsorptive transcytosis or paracellular transcytosis. As will be described herein, modified GLP-1rA's and GLP-1/GIPrA's, or pharmaceutically-acceptable salts thereof, are described which are conjugated to transcytotic carriers such as ligands, peptides or other engineered materials with affinity for high-capacity BBB receptor-mediated or non-receptor-mediated transcytotic mechanisms.

Examples of high-capacity transcytotic carriers to traverse the BBB include various iterations of the transferrin receptor (“TfR”), the folic acid receptor, CD98 herterodimeric complexes that transport amino acids, the glucose transporter 1 (Glut 1), the insulin receptor, various LDL receptors, TMEM-30A receptors, various low density lipoprotein receptor-related proteins, carbonic anhydrase 4 (Car4), insulin-like growth factors 1 and 2 (“IGF-1” and “IGF-2”) and coated artificial cell membrane structures that target and leverage BBB nicotinic acetylcholine receptors.

Generally, the described GLP-1rA's and GLP-1/GIPrA's, or pharmaceutically-acceptable salts thereof, can be coupled to any such transcytotic carrier to form GLP-1rA's and GLP-1/GIPrA's which can effectively transit through the BBB to GLP-1 and GIP receptors located within the brain. In certain embodiments, the GLP-1rA's and GLP-1/GIPrA's, or pharmaceutically-acceptable salts thereof, can be directed conjugated together with the transcytotic carrier by, for example, a chemical linkage such as a streptavidin/biotin linkage or through the construction of a fusion protein. In other embodiments, the GLP-1rA's and GLP-1/GIPrA's, or pharmaceutically-acceptable salts thereof, can be encapsulated by a liposome, polymeric nanoparticle, or solid lipid nanoparticles including transcytotic carrier ligands to allow passage of the encapsulated GLP-1rA's and GLP-1/GIPrA's through the BBB.

### Transcytotic Carriers

In certain embodiments, the transcytotic carrier can be a transporter peptide. Transporter peptides can facilitate transport of the modified GLP-1rA's and GLP-1/GIPrA's, or pharmaceutically-acceptable salts thereof, through the BBB by binding to receptors, such as neuronal nicotinic receptors (“NNRs”) or transferring receptors, located in the BBB to transfer the coupled transporter peptide and GLP-1rA's and GLP-1/GIPrA's through the BBB.

Examples of such transporter peptides that exhibit strong bonding to the binding domain of NNRs and which can facilitate BBB transcytosis include rabies virus glycoprotein (“RVG”) peptides and lynx1-loop2-derived peptides.

In certain embodiments, a suitable transporter peptide can be formed from the rabies virus glycoprotein peptide having the sequence TIWMPENPRPGTPCDIFTNSRGKRASNG (SEQ ID NO: 1) or which is known for strong binding to neuronal nicotinic acetylcholine receptors. As can be appreciated, modified peptides derived from RVG can also be suitable. For example, a modified RVG transporter peptide having additional nonamer arginine resides at the carboxy terminus (YTIWMPENPRPGTPCDIFTNSRGKRASNGGGGRRRRRRRRR (SEQ ID NO: 2)) can alternatively be used as a suitable transporter peptide. Additional RVG derived peptides that can be suitable for transport of GLP-1rA's and GLP-1/GIPrA's through the BBB can include the RVG peptides disclosed in U.S. Pat. No. 8,748,567, hereby incorporated by reference herein.

In certain embodiments, a suitable transporter peptide that can bind to NNRs to allow transit through the BBB can alternatively be a lynx1-loop2-derived peptide having the general sequence of X₁-X₂-X₃-X₄-X₅-X₆-X₇-X₈-X₉-X₁₀-R-X₁₂-K-X₁₄-X₁₅-X₁₆, (hereinafter, the “lynx1-loop2-derived peptide general sequence”) wherein:
X1 is M or T;
X2 is T or I;
X3 is T or W;
X4 is R or C;
X5 is T or D;
X6 is Y, I, or G;
X7 is F or Y;
X8 is T or C;
X9 is P, N, or S;
X10 is Y, T, or S;
X12 is M or G;
X14 is V or R;
X15 is R, S, A, or I; and
X16 is K, S, or D.

For example, suitable sequences for a transporter peptide for the lynx1-loop2-derived peptide general sequence can include MTTRTYFTPYRMKVRK (SEQ ID NO: 4), MTTRTYYTPTRMKVSK (SEQ ID NO: 5), MTWCDYFTPSRGKVRKS (SEQ ID NO: 6), and MTTRTYFTPYRGKVRK (SEQ ID NO: 7).

As can be appreciated, other transporter peptides can alternatively be suitable such as peptides that bind to transferrin receptor peptides. An example transferrin receptor peptide can have the sequence CRTIGPSVC (SEQ ID NO. 8) which is a cyclic iron-mimicking peptide. Additional transferrin receptor peptides are disclosed in U.S. Pat. No. 11,097,010 and U.S. Patent App. Pub. Nos. 2024/0131177 and 2021/0130485 each of which is herein incorporated by reference.

Yet other transcytotic carriers can be suitable. For example, suitable low-density lipoproteins are disclosed in U.S. Pat. No. 7,682,627 which is herein incorporated by reference. Other suitable transcytotic carriers can include FC5 single domain llama antibodies which can bind to TMEM-30A receptors.

Any of the transporter peptides described or referenced herein can be constructed as known in the art such as by using a peptide synthesizer or a synthesized recombinantly. Methods and materials for chemical synthesis of polypeptides are well known in the art. See, e.g., Merrifield, 1963, “Solid Phase Synthesis,” J. Am. Chem. Soc. 83:2149-2154. Alternatively, transporter peptides as described herein can be commercially obtained.

Generally, the transporter peptides disclosed herein can be modified such as to make the transporter peptides more hydrophobic or hydrophilic. For example, individual substitutions, deletions or additions that alter, add or delete a single amino acid or a small number of amino acids (e.g., about 1 to about 5 amino acids) can be made to a transporter peptide. The choice of conservative amino acids for conservative substitutions may be selected based on the location of the amino acid to be substituted in the peptide. For example, the choice can depend on whether the amino acid is on the exterior of the peptide and exposed to solvents, or on the interior and not exposed to solvents.

In certain embodiments, suitable transcytotic carriers can bind to receptors predominantly located within the BBB and not widely expressed throughout the body. Such transcytotic carriers can maximize transmission through the BBB while minimizing delivery of the GLP-1rAs or GLP-1/GIPrAs to peripheral tissue. By minimizing delivery of GLP-1rA or GLP-1/GIPrA to peripheral tissues, side effects of the GLP-1rA or GLP-1/GIPrA can thereby be minimized. In certain embodiments, such transcytotic carriers can include RGV and lynx1-loop2-derived peptides.

### Modified Constructs

In certain embodiments, the transporter peptide can be conjugated to the GLP-1rA or GLP-1/GIPrA to form a modified GLP-1rA or GLP-1/GIPrA, or a pharmaceutically-acceptable salt thereof, as described herein. In certain embodiments, the transporter peptide can have a peptide sequence that is about 70% or more identical to one or more of SEQ ID NOs: 1, 2, 4, 5, 6, 7, or the lynx1-loop2-derived peptide general sequence. In certain embodiments, the transporter peptide can have a peptide sequence that is about 80% or more identical to one or more of SEQ ID NOs: 1, 2, 4, 5, 6, 7, or the lynx1-loop2-derived peptide general sequence. In certain embodiments, the transporter peptide can have a peptide sequence that is about 90% or more identical to one or more of SEQ ID NOs: 1, 2, 4, 5, 6, 7, or the lynx1-loop2-derived peptide general sequence. In certain embodiments, the transporter peptide can have a peptide sequence that is about 95% or more identical to one or more of SEQ ID NOs: 1, 2, 4, 5, 6, 7, or the lynx1-loop2-derived peptide general sequence. In certain embodiments, the transporter peptide can have a peptide sequence that is about 100% identical to one or more of SEQ ID NOs: 1, 2, 4, 5, 6, 7, or the lynx1-loop2-derived peptide general sequence. In certain embodiments, the transporter peptide can have a peptide sequence that is identical to one or more of SEQ ID NOs: 1, 2, 4, 5, 6, 7, or the lynx1-loop2-derived peptide general sequence.

In certain embodiments, the transporter peptides can be ligands on the surface of liposomes or polymeric nanoparticles that include the GLP-1rA or GLP-1/GIPrA. In certain embodiments, the transporter peptide can have a peptide sequence that is about 70% or more identical to one or more of SEQ ID NOs: 1, 2, 4, 5, 6, 7, or the lynx1-loop2-derived peptide general sequence. In certain embodiments, the transporter peptide can have a peptide sequence that is about 80% or more identical to one or more of SEQ ID NOs: 1, 2, 4, 5, 6, 7, or the lynx1-loop2-derived peptide general sequence. In certain embodiments, the transporter peptide can have a peptide sequence that is about 90% or more identical to one or more of SEQ ID NOs: 1, 2, 4, 5, 6, 7, or the lynx1-loop2-derived peptide general sequence. In certain embodiments, the transporter peptide can have a peptide sequence that is about 95% or more identical to one or more of SEQ ID NOs: 1, 2, 4, 5, 6, 7, or the lynx1-loop2-derived peptide general sequence. In certain embodiments, the transporter peptide can have a peptide sequence that is about 100% identical to one or more of SEQ ID NOs: 1, 2, 4, 5, 6, 7, or the lynx1-loop2-derived peptide general sequence. In certain embodiments, the transporter peptide can have a peptide sequence that is identical to one or more of SEQ ID NOs: 1, 2, 4, 5, 6, 7, or the lynx1-loop2-derived peptide general sequence.

In certain embodiments, transport across the BBB can be verified by cellular imaging or confocal microscopy of brain sections of subjects injected with a modified GLP-1rA's or GLP-1/GIPrA's.

Additional examples of transporter peptides to transport the therapeutic agents disclosed herein across the BBB are disclosed in U.S. Pat. No. 12,208,143 B2, which is hereby incorporated herein by reference in its entirety.

### Therapeutic Treatments

### Purity of Compounds of the Disclosure

Any compound of the disclosure (e.g., GLP-1rA or GLP-1/GIPrA or a pharmaceutically-acceptable salt thereof) can be purified. A compound of the disclosure (e.g., GLP-1rA or GLP-1/GIPrA) can be designated a drug. A compound as described herein can be least 1% pure, at least 2% pure, at least 3% pure, at least 4% pure, at least 5% pure, at least 6% pure, at least 7% pure, at least 8% pure, at least 9% pure, at least 10% pure, at least 11% pure, at least 12% pure, at least 13% pure, at least 14% pure, at least 15% pure, at least 16% pure, at least 17% pure, at least 18% pure, at least 19% pure, at least 20% pure, at least 21% pure, at least 22% pure, at least 23% pure, at least 24% pure, at least 25% pure, at least 26% pure, at least 27% pure, at least 28% pure, at least 29% pure, at least 30% pure, at least 31% pure, at least 32% pure, at least 33% pure, at least 34% pure, at least 35% pure, at least 36% pure, at least 37% pure, at least 38% pure, at least 39% pure, at least 40% pure, at least 41% pure, at least 42% pure, at least 43% pure, at least 44% pure, at least 45% pure, at least 46% pure, at least 47% pure, at least 48% pure, at least 49% pure, at least 50% pure, at least 51% pure, at least 52% pure, at least 53% pure, at least 54% pure, at least 55% pure, at least 56% pure, at least 57% pure, at least 58% pure, at least 59% pure, at least 60% pure, at least 61% pure, at least 62% pure, at least 63% pure, at least 64% pure, at least 65% pure, at least 66% pure, at least 67% pure, at least 68% pure, at least 69% pure, at least 70% pure, at least 71% pure, at least 72% pure, at least 73% pure, at least 74% pure, at least 75% pure, at least 76% pure, at least 77% pure, at least 78% pure, at least 79% pure, at least 80% pure, at least 81% pure, at least 82% pure, at least 83% pure, at least 84% pure, at least 85% pure, at least 86% pure, at least 87% pure, at least 88% pure, at least 89% pure, at least 90% pure, at least 91% pure, at least 92% pure, at least 93% pure, at least 94% pure, at least 95% pure, at least 96% pure, at least 97% pure, at least 98% pure, at least 99% pure, at least 99.1% pure, at least 99.2% pure, at least 99.3% pure, at least 99.4% pure, at least 99.5% pure, at least 99.6% pure, at least 99.7% pure, at least 99.8% pure, or at least 99.9% pure.

In some embodiments, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% of the compound of the disclosure crosses the BBB. In some embodiments, about 50% or more of the compound of the disclosure crosses the BBB. In some embodiments, about 60% or more of the compound of the disclosure crosses the BBB. In some embodiments, about 70% or more of the compound of the disclosure crosses the BBB. In some embodiments, about 80% or more of the compound of the disclosure crosses the BBB. In some embodiments, about 90% or more of the compound of the disclosure crosses the BBB. In some embodiments, about 95% or more of the compound of the disclosure crosses the BBB.

In some embodiments, GLP-1rA or GLP-1/GIP1rA, or a pharmaceutically-acceptable salt thereof, can be administered via a route selected from a topical route (such as epicutaneous, inhalational, nasal, ophthalmic, auricular/aural, vaginal, mucosal); an enteral route (such as oral, gastrointestinal, sublingual, sublabial, buccal, rectal); and a parenteral route (such as intravenous, intraarterial, intraosseous, intramuscular, intracerebral, intracerebroventricular, epidural, intrathecal, subcutaneous, intraperitoneal, extra-amniotic, intraarticular, intracardiac, intradermal, intralesional, intrauterine, intravesical, intravitreal, transdermal, intranasal, transmucosal, intrasynovial, intraluminal). In one aspect, the administration of the method is intravenous (IV) administration. In certain embodiments, the amount and timing of administration can vary depending on factors such as the biological half-life.

### Therapeutic Methods

The present disclosure describes the use of compounds and methods to treat metabolic or CNS disorders. The disclosure also describes techniques to target the CNS with GLP-1rA or GLP-1/GIPrA while avoiding high first-pass hepatic metabolism and circumventing the BBB.

The compounds and methods of the disclosure can be used to treat metabolic disorders. Metabolic disorders include gastrointestinal (“GI”) and gastro-hormonal function disorder (including insulin secretion and insulin sensitivity) and meal related behaviors. The metabolic disorders can include the treatment of obesity and type-2 diabetes mellitus (“T2DM”). The compounds and methods of the disclosure can influence CNS components of metabolic disorders such as by affecting feeding and hunger.

The compounds and methods of the disclosure can be used to treat CNS conditions. CNS disorders are a group of neurological disorders that affect the structure or function of the brain or spinal cord, which collectively form the CNS. The disclosure describes compounds and methods to treat a CNS disorder caused by traumatic brain injury, mild traumatic brain injury, concussion, mild concussion, post-concussion syndrome, infections, degeneration (e.g., degenerative spinal disorders), structural defects (e.g., anencephaly, hypospadias, spina bifida, microgyria, polymicrogyria, bilateral frontoparietal polymicrogyria, or pachgyria), tumors, autoimmune disorders, diabetic neuropathy, or stroke. In some embodiments, the disclosure describes compounds and methods of treating the use of a compound of the disclosure to treat traumatic brain injury. In some embodiments, the disclosure describes the use of a compound of the disclosure to treat mild traumatic brain injury. In some embodiments, the disclosure describes the use of a compound of the disclosure to treat subarachnoid hemorrhage. In some embodiments, the disclosure describes the use of a compound of the disclosure to treat concussion. In some embodiments, the disclosure describes the use of a compound of the disclosure to treat a mild concussion. In some embodiments, the disclosure describes the use of a compound of the disclosure to treat post-concussion syndrome. Examples of further CNS diseases that can be treated by GLP-1rA and GLP-1/GIPrA drugs described herein include the CNS diseases disclosed in U.S. Provisional Patent App. Ser. No. 63/733,616, which is hereby incorporated by reference herein in its entirety.

### Pharmaceutical Compositions

The present disclosure describes pharmaceutical compositions comprising modified GLP-1rAs or GLP-1/GIPrAs that include conjugated transcytotic carriers to facilitate transfer through the BBB. A pharmaceutical composition of the disclosure can be a combination of any pharmaceutical compound described herein with other chemical components or therapeutic agents, such as carriers, stabilizers, diluents, dispersing agents, suspending agents, thickening agents, and/or excipients. The pharmaceutical composition facilitates administration of the compound to an organism. Pharmaceutical compositions can be administered in therapeutically-effective amounts as pharmaceutical compositions by various forms and routes including, for example, intravenous, subcutaneous, intramuscular, oral, parenteral, ophthalmic, subcutaneous, transdermal, nasal, vaginal, and topical administration. A pharmaceutical composition can be administered in a local manner, for example, via injection of the compound directly into an organ, optionally in a depot or sustained release formulation or implant.

In practicing the methods of treatment or use provided herein, therapeutically-effective amounts of a compound described herein are administered in pharmaceutical compositions to a subject having a disease or condition to be treated. In some embodiments, the subject is a mammal such as a human. A therapeutically-effective amount can vary widely depending on the severity of the disease, the age and relative health of the subject, the potency of the compound used, and other factors. A compound of the disclosure can be used singly or in combination with one or more therapeutic agents as components of mixtures. In certain embodiments, a therapeutically-effective amount is about 1 mg/kg to about 10 mg/kg. In certain embodiments, a therapeutically-effective amount is from about 100 mg to about 400 mg.

Pharmaceutical compositions of the disclosure can be formulated using one or more physiologically-acceptable carriers comprising excipients and auxiliaries, which facilitate processing of the active compounds into preparations that can be used pharmaceutically. A formulation can be modified depending upon the route of administration chosen. Pharmaceutical compositions comprising a compound described herein can be manufactured, for example, by mixing, dissolving, emulsifying, encapsulating, entrapping, or compression processes.

The pharmaceutical compositions of the disclosure can include at least one pharmaceutically-acceptable carrier, diluent, or excipient and compounds described herein as free-base or pharmaceutically-acceptable salt form. Pharmaceutical compositions can contain solubilizers, stabilizers, tonicity enhancing agents, buffers and preservatives.

Methods for the preparation of compositions comprising a compound described herein include formulating a compound with one or more inert, pharmaceutically-acceptable excipients or carriers to form a solid, semi-solid, or liquid composition. Solid compositions include, for example, powders, tablets, dispersible granules, capsules, and cachets. Liquid compositions include, for example, solutions in which a compound is dissolved, emulsions comprising a compound, or a solution containing liposomes, micelles, or nanoparticles comprising a compound as disclosed herein. Semi-solid compositions include, for example, gels, suspensions and creams. The compositions can be in liquid solutions or suspensions, solid forms suitable for solution or suspension in a liquid prior to use, or as emulsions. These compositions can also contain minor amounts of nontoxic, auxiliary substances, such as wetting or emulsifying agents, pH buffering agents, and other pharmaceutically-acceptable additives.

Non-limiting examples of dosage forms suitable for use in the disclosure include liquid, powder, gel, nanosuspension, nanoparticle, microgel, aqueous or oily suspensions, emulsion, and any combination thereof.

In some embodiments, the composition comprise adsorption enhancers. Excipients that improve permeation and absorption are e.g., cyclodextrins, bile salts, laureth-9 sulfate, fusidate derivates, fatty acids, hydrophilic polymers, and surfactants. In some embodiments, methylated (β-cyclodextrins are used to enhance the absorption of poorly water soluble, lipophilic drugs since they form inclusion complexes with the drug.

In some embodiments, the compositions are in solid or semi-sold forms, e.g., in the form of hydrogels. Examples of hydrogels include compositions comprising chitosan, carbopol, hydroxypropyl methylcellulose, and/or polyvinyl alcohol. Due to higher viscosity, semisolid formulations can target the olfactory cleft with its mucosa facing either upside-down or upright.

In some embodiments, the compositions are in particle form. In some embodiments, the compositions are in the form of nanoparticles. The use of nanoparticles as drug delivery systems allows controlled and site-specific delivery of therapeutic agents. Nanoparticles are able to protect the drug from biological or chemical degradation and help to evade drug-efflux mechanisms such as P-glycoprotein transporter in the BBB, due to encapsulation of the drug. The use of nano- and microparticles for drug delivery brings along some benefits, such as a controlled and sustained drug release or by shielding the drug against environmental influences. Nanocarriers for drug delivery systems include, but are not limited to, polymeric, lipid and inorganic nanoparticles. Biodegradable drug carriers as poly lactic acid (PLA), poly glycolic acid (PGA) and their polymer poly(lactid-co-glycolid) acid (PLGA).

In some embodiments, the compositions are lipid-based compositions. Lipid-based compositions can comprise lipid-based carriers such as mono-, di-, triglycerides, fatty acids and waxes. Nanostructured lipid carriers can be composed of blends of solid and liquid state lipids.

A composition of the disclosure can be, for example, an immediate release form. An immediate release formulation can be formulated to allow a compound to act rapidly. Non-limiting examples of immediate release formulations include readily dissolvable formulations.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions described herein can be in unit dosage forms suitable for single administration of precise dosages. In unit dosage form, the formulation is divided into unit doses containing appropriate quantities of one or more compounds. The unit dosage can be in the form of a package containing discrete quantities of the formulation. Non-limiting examples are packaged injectables, vials, or ampoules. Aqueous suspension compositions can be packaged in single-dose non-reclosable containers. Multiple-dose reclosable containers can be used, for example, in combination with or without a preservative. Formulations for injection can be presented in unit dosage form, for example, in ampoules, or in multi-dose containers with a preservative.

Pharmaceutical compositions provided herein, can be administered in conjunction with other therapies, for example, chemotherapy, radiation, surgery, anti-inflammatory agents, and selected vitamins. The other agents can be administered prior to, after, or concomitantly with the pharmaceutical compositions.

Depending on the intended mode of administration, the pharmaceutical compositions can be in the form of solid, semi-solid or liquid dosage forms, such as, for example, tablets, suppositories, pills, capsules, powders, liquids, suspensions, lotions, creams, or gels, for example, in unit dosage form suitable for single administration of a precise dosage.

For solid compositions, nontoxic solid carriers include, for example, pharmaceutical grades of mannitol, lactose, starch, magnesium stearate, sodium saccharin, talc, cellulose, glucose, sucrose, and magnesium carbonate.

Compounds can be delivered via liposomal technology. The use of liposomes as drug carriers can increase the therapeutic index of a compound. Liposomes are composed of natural phospholipids, and can contain mixed lipid chains with surfactant properties (e.g., egg phosphatidylethanolamine). A liposome design can employ surface ligands for attaching to unhealthy tissue. Non-limiting examples of liposomes include the multilamellar vesicle (MLV), the small unilamellar vesicle (SUV), and the large unilamellar vesicle (LUV). Liposomal physicochemical properties can be modulated to optimize penetration through biological barriers and retention at the site of administration, and to reduce a likelihood of developing premature degradation and toxicity to non-target tissues.

Optimal liposomal properties depend on the administration route: large-sized liposomes show good retention upon local injection, small-sized liposomes are better suited to achieve passive targeting. PEGylation reduces the uptake of the liposomes by the liver and spleen, and increases the circulation time, resulting in increased localization at the inflamed site due to the enhanced permeability and retention (EPR) effect. Additionally, liposomal surfaces can be modified to achieve selective delivery of the encapsulated drug to specific target cells. Non-limiting examples of targeting ligands include monoclonal antibodies, vitamins, peptides, and polysaccharides specific for receptors concentrated on the surface of cells associated with the disease.

Non-limiting examples of dosage forms suitable for use in the disclosure include liquid, elixir, nanosuspension, aqueous or oily suspensions, drops, syrups, and any combination thereof. Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, anti-oxidants, gums, coating agents, coloring agents, flavoring agents, coating agents, plasticizers, preservatives, suspending agents, emulsifying agents, plant cellulosic material and spheronization agents, and any combination thereof.

A pharmaceutical composition of the disclosure can be in the form of an aqueous solution. In some embodiments, the pharmaceutical composition can comprise from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, or from about 85% to about 90% of a modified GLP-1rA or GLP-1/GIPrA, or a pharmaceutically-acceptable salt thereof as described herein in an aqueous solution.

In some embodiments, the pharmaceutical composition can comprise from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, or from about 85% to about 90% of GLP-1rA or GLP-1/GIPrA, or a pharmaceutically-acceptable salt thereof in an aqueous solution.

In some embodiments, a composition of the disclosure can comprise a co-solvent. In some embodiments, the cosolvent is dimethylformamide. In some embodiments, the co-solvent is high purity dimethylformamide. In some embodiments, the cosolvent is dimethylsulfoxide. In some embodiments, a compound of the disclosure can be dissolved in a co-solvent at a concentration of from about 10 mg/mL to about 20 mg/mL, from about 20 mg/mL to about 30 mg/mL, from about 30 mg/mL to about 40 mg/mL, from about 40 mg/mL to about 50 mg/mL, or from about 50 mg/mL to about 60 mg/mL. In some embodiments, a compound of the disclosure can be dissolved in a co-solvent at a concentration of from about 30 mg/mL to about 40 mg/mL. In some embodiments, a compound of the disclosure can be dissolved in a co-solvent at a concentration of from about 40 mg/mL to about 50 mg/mL. In some embodiments, a compound of the disclosure can be dissolved in a co-solvent at a concentration from about 50 mg/mL to about 60 mg/mL. In some embodiments, a compound of the disclosure can be dissolved in a co-solvent at a concentration of about 10 mg/mL, about 20 mg/mL, about 30 mg/mL, about 40 mg/mL, about 50 mg/mL, about 60 mg/mL. In some embodiments, a compound of the disclosure can be dissolved in a co-solvent at a concentration of about 40 mg/mL. In some embodiments, a compound of the disclosure can be dissolved in a co-solvent at a concentration of about 50 mg/mL. In some embodiments, a compound of the disclosure can be dissolved in a co-solvent at a concentration of about 60 mg/mL

Compositions of the disclosure can be packaged as a kit. In some embodiments, a kit includes written instructions on the administration/use of the composition. The written material can be, for example, a label. The written material can suggest conditions or methods of administration. The instructions provide the subject and the supervising physician with the best guidance for achieving the optimal clinical outcome from the administration of the therapy. The written material can be a label. In some embodiments, the label can be approved by a regulatory agency, for example the U.S. Food and Drug Administration (FDA), the European Medicines Agency (EMA), or other regulatory agencies.

### Dosing

Pharmaceutical compositions described herein can be in unit dosage forms suitable for single administration of precise dosages. In unit dosage form, the formulation is divided into unit doses containing appropriate quantities of one or more compound. The unit dosage can be in the form of a package containing discrete quantities of the formulation. Non-limiting examples are liquids in vials or ampoules. Aqueous suspension compositions can be packaged in single-dose non-reclosable containers. Multiple-dose reclosable containers can be used, for example, in combination with a preservative. Formulations for parenteral injection can be presented in unit dosage form, for example, in ampoules, or in multi-dose containers with a preservative.

A compound described herein can be administered to a subject in a composition in a range of from, about 1 mg/kg to about 5 mg/kg, about 1 mg/kg to about 10 mg/kg, about 1 mg/kg to about 25 mg/kg, about 10 mg/kg to about 50 mg/kg, about 10 mg/kg to about 75 mg/kg, about 10 mg/kg to about 100 mg/kg, about 50 mg/kg to about 125 mg/kg, about 50 mg/kg to about 150 mg/kg, about 50 mg/kg to about 175 mg/kg, about 50 mg/kg to about 200 mg/kg, about 100 mg/kg to about 225 mg/kg, about 100 mg/kg to about 250 mg/kg, about 100 mg/kg to about 275 mg/kg, about 100 mg/kg to about 300 mg/kg, about 150 mg/kg to about 325 mg/kg, about 150 mg/kg to about 350 mg/kg, about 150 mg/kg to about 375 mg/kg, about 150 mg/kg to about 400 mg/kg, about 250 mg/kg to about 425 mg/kg, about 250 mg/kg to about 450 mg/kg, or about 250 mg/kg to about 500 mg/kg.

In some embodiments, a compound is administered in an amount ranging from about 5 mg/kg to about 50 mg/kg, about 250 mg/kg to about 2000 mg/kg, about 10 mg/kg to about 800 mg/kg, about 50 mg/kg to about 400 mg/kg, about 100 mg/kg to about 300 mg/kg, or about 150 mg/kg to about 200 mg/kg. In some embodiments, a compound described herein can be present in a composition in a range of from about 20 mg/kg to about 400 mg/kg. In some embodiments, a compound described herein can be present in a composition in a range of from about 20 mg/kg to about 240 mg/kg. In some embodiments, a compound described herein can be present in a composition in a range of from about 75 mg/kg to about 150 mg/kg. In some embodiments, a compound described herein can be present in a composition in a range of from about 75 mg/kg to about 150 mg/kg. In some embodiments, a compound described herein can be present in a composition in a range of from about 100 mg/kg to about 150 mg/kg.

In some embodiments, a compound described herein can be present in a composition in an amount of about 75 mg/kg. In some embodiments, a compound described herein can be present in a composition in an amount of about 100 mg/kg. In some embodiments, a compound described herein can be present in a composition in an amount of about 150 mg/kg. In some embodiments, a compound described herein can be present in a composition in an amount of about 200 mg/kg. In some embodiments, a compound described herein can be present in a composition in an amount of about 250 mg/kg. In some embodiments, a compound described herein can be present in a composition in an amount of about 400 mg/kg.

A compound described herein can be present in a composition in a range of from about 1 mg to about 2000 mg, from about 100 mg to about 2000 mg, from about 10 mg to about 2000 mg, from about 5 mg to about 1000 mg, from about 10 mg to about 500 mg, from about 50 mg to about 250 mg, from about 100 mg to about 200 mg, from about 1 mg to about 50 mg, from about 50 mg to about 100 mg, from about 100 mg to about 150 mg, from about 150 mg to about 200 mg, from about 200 mg to about 250 mg, from about 250 mg to about 300 mg, from about 300 mg to about 350 mg, from about 350 mg to about 400 mg, from about 400 mg to about 450 mg, from about 450 mg to about 500 mg, from about 500 mg to about 550 mg, from about 550 mg to about 600 mg, from about 600 mg to about 650 mg, from about 650 mg to about 700 mg, from about 700 mg to about 750 mg, from about 750 mg to about 800 mg, from about 800 mg to about 850 mg, from about 850 mg to about 900 mg, from about 900 mg to about 950 mg, or from about 950 mg to about 1000 mg. In some embodiments, a method administers a therapeutically-effective amount from about 100 mg to about 400 mg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a compound described herein can be present in a composition in an amount of about 100 mg, about 120 mg, about 140 mg, about 160 mg, about 180 mg, about 200 mg, about 220 mg, about 240 mg, about 260 mg, about 280 mg, or about 300 mg. In some embodiments, a compound described herein can be present in a composition in an amount of about 150 mg. In some embodiments, a compound described herein can be present in a composition in an amount of about 170 mg. In some embodiments, a compound described herein can be present in a composition in an amount of about 280 mg. In some embodiments, a compound described herein can be present in a composition in an amount of about 300 mg. In some embodiments, a compound described herein can be present in a composition in an amount of about 350 mg. In some embodiments, a compound described herein can be present in a composition in an amount of about 400 mg.

In some embodiments, a compound of the disclosure can be administered to a subject in a composition in an amount of about 1 mg/kg to about 10 mg/kg. In some embodiments, a compound of the disclosure can be administered to a subject in a composition in an amount of about 1 mg/kg to about 50 mg/kg. In some embodiments, a compound of the disclosure can be administered to a subject in a composition in an amount of about 1 mg/kg to about 75 mg/kg. In some embodiments, a compound of the disclosure can be administered to a subject in a composition in an amount of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, or about 10 mg/kg.

A compound described herein can be present in a composition in an amount of about 1 mg, about 2 mg, about 3 mg, about 4 mg, about 5 mg, about 10 mg, about 15 mg, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 125 mg, about 150 mg, about 175 mg, about 200 mg, about 250 mg, about 300 mg, about 350 mg, about 400 mg, about 450 mg, about 500 mg, about 550 mg, about 600 mg, about 650 mg, about 700 mg, about 750 mg, about 800 mg, about 850 mg, about 900 mg, about 950 mg, about 1000 mg, about 1050 mg, about 1100 mg, about 1150 mg, about 1200 mg, about 1250 mg, about 1300 mg, about 1350 mg, about 1400 mg, about 1450 mg, about 1500 mg, about 1550 mg, about 1600 mg, about 1650 mg, about 1700 mg, about 1750 mg, about 1800 mg, about 1850 mg, about 1900 mg, about 1950 mg, or about 2000 mg.

In some embodiments, a dosage is administered in each nostril in about equal amounts. In some embodiments, about 0.1 mL, about 0.2 mL, about 0.3 mL, about 0.4 mL, about 0.5 mL, about 0.6 mL, about 0.7 mL, about 0.8 mL, about 0.9 mL, or about 1.0 mL of a pharmaceutical composition is administered in each nostril of a subject. In some embodiments, 0.25 mL of a pharmaceutical composition is administered in each nostril per dose. In some embodiments, 0.5 mL of a pharmaceutical composition is administered in each nostril per dose.

In some embodiments, dosing is repeated for each nostril to increase the amount of a compound. In some embodiments, a first dosage of a pharmaceutical composition is about 0.1 mL, about 0.2 mL, about 0.3 mL, about 0.4 mL, about 0.5 mL, about 0.6 mL, about 0.7 mL, about 0.8 mL, about 0.9 mL, or about 1.0 mL per nostril of the subject. In some embodiments, a follow up dosage is the same amount as the first dosage of the pharmaceutical composition is. In some embodiments, a follow up dosage is a different amount as the first dosage of the pharmaceutical composition is. In some embodiments, the follow up dosage is a smaller amount than the first dosage of the pharmaceutical composition is. In some embodiments, the follow up dosage is a greater amount than the first dosage of the pharmaceutical composition is.

Therapeutic agents described herein can be administered before, during, or after the occurrence of a disease or condition, and the timing of administering the composition containing a therapeutic agent can vary. In some embodiments, the compositions can be used as a prophylactic and can be administered continuously to subjects with a propensity to conditions or diseases to lessen a likelihood of the occurrence of the disease or condition. The compositions can be administered to a subject during or as soon as possible after the onset of the symptoms. The administration of the therapeutic agents can be initiated within the first 48 hours of the onset of the symptoms, within the first 24 hours of the onset of the symptoms, within the first 6 hours of the onset of the symptoms, or within 3 hours of the onset of the symptoms. The initial administration can be via any route practical, such as by any route described herein using any formulation described herein. A therapeutic agent can be administered as soon as is practicable after the onset of a disease or condition is detected or suspected, and for a length of time necessary for the treatment of the disease, such as, for example, from about 1 month to about 3 months. The length of treatment can vary for each subject.

A compound or pharmaceutical composition of the disclosure can be administered more than one time. In some embodiments, a compound or pharmaceutical composition of the disclosure can be administered once daily. In some embodiments, a compound or pharmaceutical composition of the disclosure can be administered twice daily. In some embodiments, a compound or pharmaceutical composition of the disclosure can be administered three times daily. In some embodiments, a compound or pharmaceutical composition of the disclosure can be administered, and the administration can be repeated at least once. In some embodiments, administration of a compound or a pharmaceutical composition can be repeated once. In some embodiments, administration of a compound or a pharmaceutical composition can be repeated twice. In some embodiments, administration of a compound or a pharmaceutical composition can be repeated three times.

In some embodiments, administration of a compound or a pharmaceutical composition can be repeated after about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, about 21 days, about 22 days, about 23 days, about 24 days, about 25 days, about 26 days, about 27 days, about 28 days, about 29 days, about 30 days, or about 31 days. In some embodiments, administration of a compound or pharmaceutical composition can be repeated after about 7 days. In some embodiments, administration of a compound or pharmaceutical composition can be repeated after about 14 days.

### Combination Therapy

The compounds of the disclosure can be administered with at least one additional therapeutic agent. In some embodiments, the compounds of the disclosure can be administered with one additional therapeutic agent. In some embodiments, the compounds of the disclosure can be administered with two additional therapeutic agents. In some embodiments, the compounds of the disclosure can be administered with three additional therapeutic agents.

In some embodiments, a compound of the invention is administered in combination with, before, or after treatment with another therapeutic agent. If simultaneously, the multiple therapeutic agents can be provided in a single, unified form, or in multiple forms, for example, as multiple separate pills. The agents can be packed together or separately, in a single package or in a plurality of packages. One or all of the therapeutic agents can be given in multiple doses. If not simultaneous, the timing between the multiple doses can vary to as much as about a month.

In some embodiments, administering GLP-1rA or GLP-1/GIPrA can increase the delivery of an additional therapeutic agent. In some embodiments, GLP-1rA or the GLP-1/GIPrA can act as a promoter to transfer an additional therapeutic agent.

In some embodiments, administering GLP-1rA or GLP-1/GIPrA with an additional therapeutic agent can increase the delivery of the additional therapeutic agent by at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 100%, at least about 110%, at least about 120%, at least about 130%, at least about 140%, at least about 150%, at least about 160%, at least about 170%, at least about 180%, at least about 190%, or at least about 200% as compared to delivery of the additional therapeutic agent alone. In some embodiments, administering GLP-1rA or GLP-1/GIPrA with an additional therapeutic agent can increase the delivery of the additional therapeutic agent by at least about 2-fold, at least about 2.5-fold, at least about 3-fold, at least about 3.5-fold, at least about 4-fold, at least about 4.5-fold, at least about 5-fold, at least about 5.5-fold, at least about 6-fold, at least about 6.5-fold, at least about 7-fold, at least about 7.5-fold, at least about 8-fold, at least about 8.5-fold, at least about 9-fold, at least about 9.5-fold, or at least about 10-fold as compared to delivery of the therapeutic agent alone. In some embodiments, administering GLP-1rA or GLP-1/GIPrA with an additional therapeutic agent can increase the delivery of the additional therapeutic agent by at least about 30%. In some embodiments, administering GLP-1rA or GLP-1/GIPrA with an additional therapeutic agent can increase the delivery of the additional therapeutic agent by at least about 50%. In some embodiments, administering GLP-1rA or GLP-1/GIPrA with an additional therapeutic agent can increase the delivery of the additional therapeutic agent by at least about 2-fold. In some embodiments, administering GLP-1rA or GLP-1/GIPrA with an additional therapeutic agent can increase the delivery of the additional therapeutic agent by at least about 5-fold.

In some embodiments, GLP-1rA or GLP-1/GIPrA can be administered with from about 1 mg to about 25 mg, from about 25 mg to about 50 mg, from about 50 mg to about 75 mg, from about 75 mg to about 100 mg, from about 100 mg to about 125 mg, from about 125 mg to about 150 mg, from about 150 mg to about 175 mg, from about 175 mg to about 200 mg, from about 225 mg to about 250 mg, from about 250 mg to about 275 mg, or from about 275 mg to about 300 mg of an additional therapeutic agent to increase the delivery amount of the additional therapeutic agent. In some embodiments GLP-1rA or GLP-1/GIPrA can be administered with from about 1 mg to about 25 mg of an additional therapeutic agent to increase the delivery amount of the additional therapeutic agent. In some embodiments, GLP-1rA or GLP-1/GIPrA can be administered with from about 100 mg to about 125 mg of an additional therapeutic agent to increase the delivery amount of the additional therapeutic agent.

In some embodiments, GLP-1rA or GLP-1/GIPrA can be administered with about 5 mg, about 10 mg, about 20 mg, about 30 mg, about 40 mg, about 50 mg, about 60 mg, about 70 mg, about 80 mg, about 90 mg, about 100 mg, about 120 mg, about 140 mg, about 160 mg, about 180 mg, about 200 mg, about 220 mg, about 240 mg, about 260 mg, about 280 mg, or about 300 mg of an additional therapeutic agent to increase the delivery amount of the additional therapeutic agent. In some embodiments, GLP-1rA or GLP-1/GIPrA can be administered with about 50 mg of an additional therapeutic agent to increase the delivery amount of the additional therapeutic agent. In some embodiments, GLP-1rA or GLP-1/GIPrA can be administered with about 150 mg of an additional therapeutic agent to increase the delivery amount of the additional therapeutic agent.

In some embodiments, GLP-1rA or GLP-1/GIPrA can be administered with an additional therapeutic agent at a ratio of about 1:10, about 1:9, about 1:8, about 1:7, about 1:6, about 1:5, about 1:4, about 1:3, about 1:2, about 1:1, about 2:1, about 3:1, about 4:1, about 5:1, about 6:1, about 7:1, about 8:1, about 9:1, or about 10:1 to increase the delivery amount of the additional therapeutic agent. In some embodiments, GLP-1rA or GLP-1/GIPrA can be administered with an additional therapeutic agent at a ratio of about 1:10 to increase the delivery amount of the additional therapeutic agent. In some embodiments, GLP-1rA or GLP-1/GIPrA can be administered with an additional therapeutic agent at a ratio of about 1:1 to increase the delivery amount of the additional therapeutic agent. In some embodiments, GLP-1rA or GLP-1/GIPrA can be administered with an additional therapeutic agent at a ratio of about 2:1 to increase the delivery amount of the additional therapeutic agent. In some embodiments, GLP-1rA or GLP-1/GIPrA can be administered with an additional therapeutic agent at a ratio of about 5:1 to increase the delivery amount of the additional therapeutic agent.

In some embodiments, the additional therapeutic agent is an antibacterial agent. In some embodiments, the antibacterial agent is a CNS antibacterial agent, for example betalactam or an aminoglycoside. In some embodiments, the betalactam is a penicillin, cephalosporin, monobactam, or carbapenem. In some embodiments, the aminoglycoside is gentamicin, amikacin, tobramycin, neomycin, or streptomycin. In some embodiments, the additional therapeutic agent is ambroxol. In some embodiments, the additional therapeutic agent is an antibody. In some embodiments, the antibody is aducanumab.

### Therapeutic Effects

Administration of the GLP-1rA or the GLP-1/GIPrA can change the concentration of such compounds in the brain region. In certain embodiments, the brain region is the cerebrum, brainstem, cerebellum, pons, medulla, frontal lobe, parietal lobe, occipital lobe, temporal lobe, left dorsal striatum, occipital cortex, or dorsolateral prefrontal cortex (DLPF). In some embodiments, the brain region is the occipital lobe. In some embodiments, the brain region is the occipital cortex. In some embodiments, the brain region is the cerebellum. In some embodiments, the brain region is the DLPF.

In some embodiments, the administering increases the concentration of one or more of GLP-1rA or the GLP-1/GIPrA in the brain region by from about 20% to about 300%. In some embodiments, the administering increases the concentration of one or more of GLP-1rA or the GLP-1/GIPrA in the brain region by from about 5% to about 10%, from about 10% to about 15%, from about 15% to about 20%, from about 20% to about 25%, from about 25% to about 30%, from about 30% to about 35%, from about 35% to about 40%, from about 40% to about 45%, from about 45% to about 50%, from about 50% to about 55%, from about 55% to about 60%, from about 60% to about 65%, from about 65% to about 70%, from about 70% to about 75%, from about 75% to about 80%, from about 80% to about 85%, from about 85% to about 90%, from about 90% to about 95%, or from about 95% to about 100%. In some embodiments, the administering increases the concentration of one or more of GLP-1rA or the GLP-1/GIPrA in the brain region by from about 100% to about 110%, from about 110% to about 120%, from about 120% to about 140%, from about 140% to about 160%, from about 160% to about 180%, from about 180% to about 200%, from about 200% to about 220%, from about 220% to about 240%, from about 240% to about 260%, from about 260% to about 280%, or from about 280% to about 300%.

In some embodiments, the administering increases the concentration of one or more of GLP-1rA or the GLP-1/GIPrA in the brain region by about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 210%, about 220%, about 230%, about 240%, about 250%, about 260%, about 270%, about 280%, about 290%, or about 300%. In some embodiments, the administering increases the concentration of one or more of GLP-1rA or the GLP-1/GIPrA in the brain region by about 20%. In some embodiments, the administering increases the concentration of one or more of GLP-1rA or the GLP-1/GIPrA in the brain region by about 50%. In some embodiments, the administering increases the concentration of one or more of GLP-1rA or the GLP-1/GIPrA in the brain region by about 100%. In some embodiments, the administering increases the concentration of one or more of GLP-1rA or the GLP-1/GIPrA in the brain region by about 150%. In some embodiments, the administering increases the concentration of one or more of GLP-1rA or the GLP-1/GIPrA in the brain region by about 200%.

In some embodiments, the administering of GLP-1rA or the GLP-1/GIPrA modulates the GLP-1rA or the GLP-1/GIPrA to water ratio in a brain region. In some embodiments, the administering of GLP-1rA or the GLP-1/GIPrA modulates the GLP-1rA or the GLP-1/GIPrA/water ratio in a brain region. In some embodiments, the administering of GLP-1rA or the GLP-1/GIPrA increases the water ratio in a brain region.

The dimensions and values disclosed herein are not to be understood as being strictly limited to the exact numerical values recited. Instead, unless otherwise specified, each such dimension is intended to mean both the recited value and a functionally equivalent range surrounding that value.

It should be understood that every maximum numerical limitation given throughout this specification includes every lower numerical limitation, as if such lower numerical limitations were expressly written herein. Every minimum numerical limitation given throughout this specification will include every higher numerical limitation, as if such higher numerical limitations were expressly written herein. Every numerical range given throughout this specification will include every narrower numerical range that falls within such broader numerical range, as if such narrower numerical ranges were all expressly written herein.

Every document cited herein, including any non-patent literature, cross-referenced or related patents or applications, are hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests, or discloses any such invention. Further, to the extent that any meaning or definition of a term in this document conflicts with any meaning or definition of the same term in a document incorporated by reference, the meaning or definition assigned to that term in the document shall govern.1

The foregoing description of embodiments and examples has been presented for purposes of description. It is not intended to be exhaustive or limiting to the forms described. Numerous modifications are possible in light of the above teachings. Some of those modifications have been discussed and others will be understood by those skilled in the art. The embodiments were chosen and described for illustration of various embodiments. The scope is, of course, not limited to the examples or embodiments set forth herein, but can be employed in any number of applications and equivalent articles by those of ordinary skill in the art. Rather it is hereby intended the scope be defined by the claims appended hereto.

It should be understood that certain aspects, features, structures, or characteristics of the various embodiments can be interchanged in whole or in part. Reference to certain embodiments mean that a particular aspect, feature, structure, or characteristic described in connection with certain embodiments can be included in at least one embodiment and may be interchanged with certain other embodiments. The appearances of the phrase “in certain embodiments” in various places in specification are not necessarily all referring to the same embodiment, nor are certain embodiments necessarily mutually exclusive of other certain embodiments. It should also be understood that the steps of the methods set forth herein are not necessarily required to be performed in the orders described, and the order of the steps of such methods should be understood to be merely exemplary. Likewise, additional steps can be included in such methods, and certain steps may be omitted or combined, in methods consistent with certain embodiments.

What is claimed is:

1. A compound to treat a metabolic or central nervous system disease, the compound comprising:
a transcytotic carrier conjugated to a glucagon-like-peptide-1 receptor agonist (“GLP-1rA”), a glucagon-like-peptide-1 and glucose-dependent insulinotropic polypeptide receptor dual agonist (“GLP-1/GIPrA”), or a pharmaceutically-acceptable salt thereof.

2. The compound of claim 1, wherein the transcytotic carrier is conjugated through a streptavidin/biotin linkage to the GLP-1rA, GLP-1/GIPrA, or a pharmaceutically-acceptable salt thereof.

3. The compound of claim 1, wherein the transcytotic carrier is conjugated through a fusion protein to the GLP-1rA, GLP-1/GIPrA, or a pharmaceutically-acceptable salt thereof.

4. The compound of claim 1, wherein the transcytotic carrier is conjugated to a liposome, polymeric nanoparticle, or solid lipid nanoparticle, and
wherein the GLP-1rA, GLP-1/GIPrA, or a pharmaceutically-acceptable salt thereof is encapsulated by the liposome, polymeric nanoparticle, or solid lipid nanoparticle.

5. The compound of claim 1, wherein the transcytotic carrier comprises a ligand or a peptide.

6. The compound of claim 1, wherein the transcytotic carrier binds to a receptor predominantly located within the blood-brain barrier and not widely expressed throughout peripheral tissue.

7. The compound of claim 1, wherein the transcytotic carrier comprises a transferrin receptor (“TfR”), a folic acid receptor, a CD98 herterodimeric complex, a glucose transporter 1 (Glut 1) peptide, an insulin receptor, a low-density lipoprotein receptor (“LDL receptor”) or related protein, a TMEM-30A receptor, a carbonic anhydrase 4 (Car4) receptor, an insulin-like growth factors 1 and 2 (“IGF-1” and “IGF-2”) receptor, or a nicotinic acetylcholine receptor.

8. The compound of claim 7, wherein the transcytotic carrier comprises a rabies virus glycoprotein (“RVG”) peptide or a lynx1-loop2-derived peptide.

9. The compound of claim 8, wherein the transcytotic carrier comprises the RVG glycoprotein having a sequence about 70% or more identical to:
TIWMPENPRPGTPCDIFTNSRGKRASNG;
or
YTIWMPENPRPGTPCDIFTNSRGKRASNGGGGRRRRRRRRR.

10. The compound of claim 8, wherein the transcytotic carrier comprises the lynx1-loop2-derived peptide having a sequence about 70% or more identical to X₁-X₂-X₃-X₄-X₅-X₆-X₇-X₈-X₉-X₁₀-R-X₁₂-K-X₁₄-X₁₅-X₁₆, wherein:
X1 is M or T;
X2 is T or I;
X3 is T or W;
X4 is R or C;
X5 is T or D;
X6 is Y, I, or G;
X7 is F or Y;
X8 is T or C;
X9 is P, N, or S;
X10 is Y, T, or S;
X12 is M or G;
X14 is V or R;
X15 is R, S, A, or I; and
X16 is K, S, or D.

11. The compound of claim 7, wherein the transcytotic carrier comprises the transferrin receptor peptide, the transferrin receptor peptide comprising a sequence about 70% or more identical to CRTIGPSVC.

12. A pharmaceutical composition comprising:
a therapeutically-effective amount of a compound of claim 1; and
a pharmaceutically-acceptable carrier, diluent, or excipient.

13. A method of treating a metabolic disease or a central nervous system disease, the method comprising:
topically, enterally, or parenterally administering to an individual in need a modified glucagon-like-peptide-1 receptor agonist (“GLP-1rA”), a modified glucagon-like-peptide-1 and glucose-dependent insulinotropic polypeptide receptor dual agonist (“GLP-1/GIPrA”), or a pharmaceutically-acceptable salt thereof; and
wherein the modified GLP-1rA, the modified GLP-1/GIPrA, or the pharmaceutically-acceptable salt thereof is configured to transit the blood-brain barrier.

14. The method of claim 13, wherein the modified GLP-1rA, the modified GLP-1/GIPrA, or the pharmaceutically-acceptable salt thereof is conjugated to the transcytotic carrier.

15. The method of claim 13, wherein the transcytotic carrier is conjugated through a streptavidin/biotin linkage or a fusion protein.

16. The method of claim 13, further comprising the administration of an additional therapeutic agent to the individual in need.

17. The method of claim 16, wherein the additional therapeutic agent comprises a 5-lipogenase-activating protein (FLAP) inhibitor, a Cathepsin B inhibitor, poly(ADP-ribose) polymerase (PARP) inhibitor, probenecid, phenserine, or a dopaminergic agent.

18. The method of claim 13, further comprising the administration of one or more carriers, stabilizers, diluents, dispersing agents, suspending agents, thickening agents, or excipients to the individual in need.

19. The method of claim 13, wherein the central nervous system disease comprises a neurodegenerative disease, a traumatic brain injury, a concussion, an infection, a degeneration, a structural defect, a tumor, an autoimmune disorder, a stroke, or a brain hemorrhage.

20. The method of claim 13, wherein the metabolic disease comprises diabetes or obesity.
